(19) United States
(12) Patent Application Publication
(10) Pub. No.: US 2026/0108315 A1
(43) Pub. Date: Apr. 23, 2026

(54) CONTROLLING A SURGICAL ROBOT DURING A SERVICE MODE

(71) Applicant: CMR Surgical Limited, Cambridge (GB)

(72) Inventors: Edward James Wildin Tucker, Cambridge (GB); Andrew John Howe, Cambridge (GB); Samuel Latham Bailey, Cambridge (GB); Christopher Michael Wastell, Cambridge (GB); Johnathan Man Chiu Hung, Cambridge (GB)

(73) Assignee: CMR Surgical Limited, Cambridge (GB)

(21) Appl. No.: 19/154,438

(22) PCT Filed: Dec. 20, 2023
(86) PCT No.: PCT/GB2023/053326

§ 371 Date: Aug. 7, 2025

(30) Foreign Application Priority Data
Feb. 21, 2023 (GB) 2302466.4

(51) Int. Cl. A61B 34/37; A61B 34/30

(52) U.S. Cl. CPC A61B 34/37; A61B 2034/302

(57) ABSTRACT

A control system for implementing a safety mechanism for a surgical robot, the surgical robot operable in a clinical mode in which the surgical robot can perform surgery in response to inputs received at a surgeon input device of a remote surgeon console, the clinical mode comprising safety features, and the surgical robot operable in a service mode in which tests and diagnostics can be run on the surgical robot in response to inputs received from a service interface, the service mode mirroring the clinical mode except in that several of the safety features of the clinical mode are disabled, the control system configured to, in the safety mechanism: receive a transition command to transition the surgical robot to a surgical clinical mode or a calibration clinical mode in which the surgical robot is calibrated for surgery; in response to receiving the transition command, determine if the surgical robot is operating in the service mode; and if the surgical robot is determined to be operating in the service mode, prevent the surgical robot from being transitioned to the surgical clinical mode or the calibration clinical mode.

### BACKGROUND

It is known to use robots for assisting and performing surgery. FIG. 1 illustrates a typical surgical robotic system. A surgical robot 100 consists of a base 102, an arm 104 and an instrument 106. The base supports the robot and may itself be attached rigidly to a support structure, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation. The end effector engages in a surgical procedure.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives inputs from the surgeon input device(s) 114 and converts these to control signals to move the joints of the robot arm 104 and instrument 106. The control system sends these control signals to the robot, where the corresponding joints are driven accordingly.

The surgical robotic system is operable in a clinical mode and in a service mode. The clinical mode is used by operating room staff to prepare the surgical robot for surgery and to perform surgery. The service mode is used by engineers to service the surgical robotic system, for example to run tests and diagnostics on the system or to perform software upgrades. The functionality of the surgical robotic system in the service mode can be the same as that of the clinical mode. However, the service mode enables changes to be made to the configuration and parameters of the surgical robotic system. These changes are generally made for the purpose of testing the surgical robotic system for development purposes. To enable this testing to occur unimpeded, several safety features of the clinical mode are disabled in the service mode. As a result of this, the surgical robotic system in the service mode is typically not granted regulatory approval to be used to perform surgery. The surgical robotic system must be transitioned to the clinical mode in order to be used to perform surgery within the permissions granted by the regulatory bodies.

However, because the same functionality is accessible in the service mode as in the clinical mode, it is possible to perform surgery in the service mode. Thus, if engineers forget to transition the surgical robotic system to the clinical mode after they have serviced it, there is a risk that the surgical robotic system could subsequently unintentionally be used to perform surgery whilst some or all of it is in the service mode. The part of it which is in the service mode could have a configuration which is significantly different to the predetermined configuration of the clinical mode. Usage of the surgical robotic system in the service mode for performing surgery would contravene the regulatory approval granted for the surgical robotic system, and depending on the test configuration applied to it, potentially put the patient at risk.

### SUMMARY OF THE INVENTION

According to an aspect of the invention, there is provided a control system for implementing a safety mechanism for a surgical robot, the surgical robot operable in a clinical mode in which the surgical robot can perform surgery in response to inputs received at a surgeon input device of a remote surgeon console, the clinical mode comprising safety features, and the surgical robot operable in a service mode in which tests and diagnostics can be run on the surgical robot in response to inputs received from a service interface, the service mode mirroring the clinical mode except in that several of the safety features of the clinical mode are disabled, the control system configured to, in the safety mechanism: receive a transition command to transition the surgical robot to a surgical clinical mode or a calibration clinical mode in which the surgical robot is calibrated for surgery; in response to receiving the transition command, determine if the surgical robot is operating in the service mode; and if the surgical robot is determined to be operating in the service mode, prevent the surgical robot from being transitioned to the surgical clinical mode or the calibration clinical mode.

The surgical robot may comprise a surgical robot arm. The surgical robot arm may comprise a series of joints by which its configuration can be altered, the series of joints extending from a base at a proximal end of the surgical robot arm to an attachment for a surgical instrument at a distal end of the surgical robot arm, the control system being configured to control the surgical robot arm in dependence on inputs received at the surgeon input device of the remote surgeon console, to alter the configuration of the surgical robot arm whilst maintaining an intersection between a surgical instrument attached to the surgical robot arm and a fulcrum, the control system configured to determine the position of the fulcrum in the calibration clinical mode.

The control system may be configured to transition the surgical robot to the surgical clinical mode from the calibration clinical mode only upon successful calibration of the surgical robot arm in the calibration clinical mode.

The control system may be further configured to output a signal to the remote surgeon console for output by the remote surgeon console, the signal indicative that the surgical robot has been prevented from transitioning to the surgical clinical mode or the calibration clinical mode.

The control system may be further configured to output a signal to the surgical robot for output by the surgical robot, the signal indicative that the surgical robot has been prevented from transitioning to the surgical clinical mode or the calibration clinical mode.

The transition command may be received via a user input on the surgical robot arm or via a user input on the remote surgeon console.

The control system may be further configured to, if the surgical robot is determined to not be operating in the service mode, enable the surgical robot to be transitioned to the surgical clinical mode or the calibration clinical mode.

The control system may be further configured to: receive a power cycle command to power cycle the surgical robot; in response to receiving the power cycle command, power cycling the surgical robot; upon successfully power cycling the surgical robot, enabling the clinical mode; and on subsequently receiving the transition command, enabling the surgical robot to be transitioned to the surgical clinical mode or the calibration clinical mode.

Power cycling the surgical robot may comprise performing power on self-test checks.

Power cycling the surgical robot may comprise loading the surgical robot in a predetermined configuration having predefined states.

The control system may be distributed and comprises a surgical robot controller, a surgeon input device controller, and one or more additional system controllers. The control system may be configured to: receive an engagement command to engage a surgical instrument; in response to receiving the engagement command, determine if the surgeon input device controller is operating in the service mode; and if the surgeon input device controller is determined to be operating in the service mode, prevent the surgeon input device controller from enabling the surgeon input device to control the surgical instrument.

The control system may be configured to: in response to receiving the engagement command, determine if the one or more additional system controllers is operating in the service mode; and if the one or more additional system controllers is determined to be operating in the service mode, preventing the one or more additional system controllers from enabling the surgeon input device to control the surgical instrument.

The control system may be further configured to output a signal to the remote surgeon console for output by the remote surgeon console, the signal indicative that the surgeon input device has been prevented from controlling the surgical instrument.

The control system may be further configured to output a signal to the surgical robot for output by the surgical robot, the signal indicative that the surgeon input device has been prevented from controlling the surgical instrument.

The control system may be further configured to, if the surgeon input device controller and/or the one or more additional system controllers is determined to not be operating in the service mode, enable the surgeon input device to control the surgical instrument.

The control system may be further configured to: receive a power cycle command to power cycle the surgeon input device controller and/or the one or more additional system controllers; in response to receiving the power cycle command, power cycling the surgeon input device controller and/or the one or more additional system controllers; upon successfully power cycling the surgeon input device controller and/or the one or more additional system controllers, enabling the clinical mode; and on subsequently receiving the engagement command, enabling the surgeon input device to control the surgical instrument.

Power cycling the surgeon input device controller and/or the one or more additional system controllers may comprise loading the surgeon input device controller and/or the one or more additional system controllers in a predetermined configuration having predefined states.

Each controller of the distributed control system may be operable in either the service mode or clinical mode independently of whether another controller of the distributed control system is in the service mode or clinical mode.

In the service mode, a safety feature of the clinical mode which may be disabled is fault checking on communication links between a robot arm base controller located at the base of the robot arm and joint controllers located at the joints of the robot arm.

### BRIEF DESCRIPTION OF THE FIGURES

The present invention will now be described by way of example with reference to the accompanying drawings. In the drawings:

FIG. 1 illustrates a surgical robot system for performing a surgical procedure;

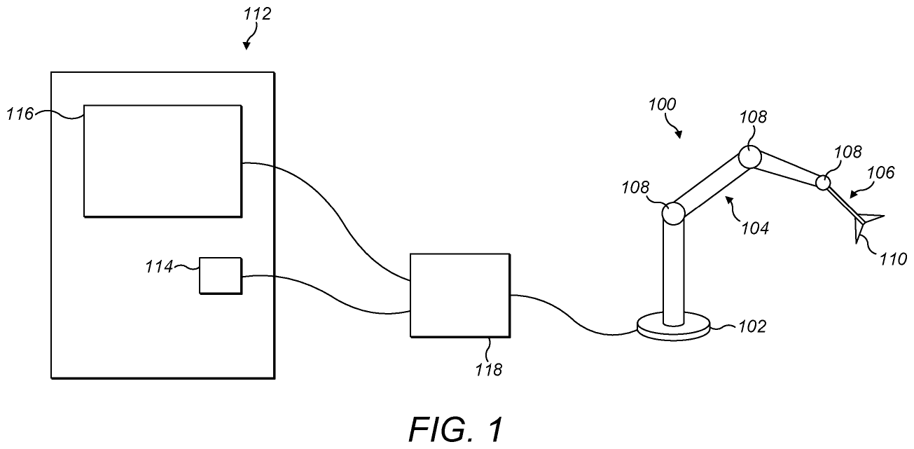

FIG. 2 illustrates a surgical robotic system;

FIG. 3 illustrates a surgical robot of a surgical robotic system;

FIG. 4 illustrates an exploded view of the joints of the surgical robot arm of FIG. 3;

FIG. 5 is a schematic diagram illustrating the control system of a surgical robotic system;

FIG. 6 illustrates operating modes of a surgical robot arm and the transitions permitted between them;

FIG. 7 is a flowchart illustrating the steps of a safety mechanism to prevent surgery taking place in the service mode; and

FIG. 8 is a flowchart illustrating the steps of a safety mechanism to prevent the surgeon input devices engaging with a surgical instrument or endoscope in the service mode.

### DETAILED DESCRIPTION

The following describes a method of controlling a surgical robotic system whilst in a service mode. In particular, safety mechanisms implemented to ensure that the surgical robotic system is not used to perform surgery whilst all or any part of it is in a service mode are discussed.

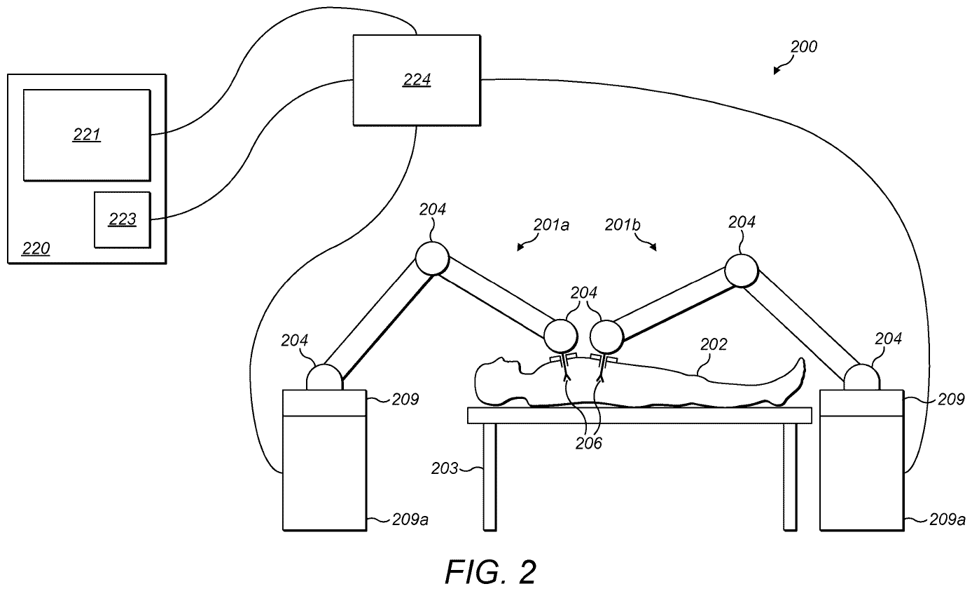

FIG. 2 shows a surgical robotic system 200 performing a surgical procedure on a patient 202. The patient 202 is positioned on an operating table 203. The surgical robotic system 200 comprises a first robot arm 201a and a second robot arm 201b. Although two robot arms 201a, 201b are shown in FIG. 2, it is to be understood that a surgical robotic system configured in accordance with the principles described herein may comprise any number of robot arms. Each robot arm 201a, 201b extends from a base 209 at its proximal end, and comprises a plurality of joints 204 by which the configuration of that robot arm can be altered. The base 209 of each robot arm 201a, 201b is supported by a support structure 209a. The support structure 209a may be moveable, for example a cart or trolley.

Each robot arm 201a, 201b comprises an attachment for a surgical instrument 206 at its distal end. The surgical instrument may have a thin elongate shaft with an end effector at its distal end for manipulating tissue of a patient at a surgical site. The surgical instrument could, for example, be a pair of jaws, scalpel, suturing needle etc. Alternatively, the surgical instrument may be an endoscope having a camera at its distal end for capturing a video feed of a surgical site. Each surgical instrument 206 is insertable into a patient's body 202, for example through a port. Different surgical instruments may access the surgical site through the same port or through different ports.

The configuration of each robot arm 201a, 201b may be remotely controlled in response to inputs received at a remote surgeon console 220. A surgeon may provide inputs to the remote console 220. The remote surgeon console comprises one or more surgeon input devices 223. For example, these may take the form of a hand controller, a foot controller such as a pedal, a touch sensitive input to be controlled by a finger or another part of the body, a voice control input device, an eye control input device or a gesture control input device. The surgeon input device may provide several inputs which the surgeon can individually operate. Each surgeon input device may be used to control a different surgical instrument. Thus, for example, a surgeon may control one surgical instrument using a hand controller in his left hand, and control another surgical instrument using a hand controller in his right hand. The surgeon console also comprises a display 221.

A control system 224 connects the surgeon console 220 to each surgical robot 201a, 201b. The control system receives inputs from the surgeon input device(s) and converts these to control signals to move the joints of the robot arms 104 and surgical instrument 206. The control system 224 sends these control signals to the robot, where the corresponding joints are driven accordingly.

The configuration of each robot arm 201a, 201b may be controllable in response to external forces applied directly to that robot arm. For example, a member of the bedside team (e.g. an operating room nurse) may apply forces directly to a robot arm (e.g. by pushing a joint of the robot arm). This behaviour will be described in further detail herein.

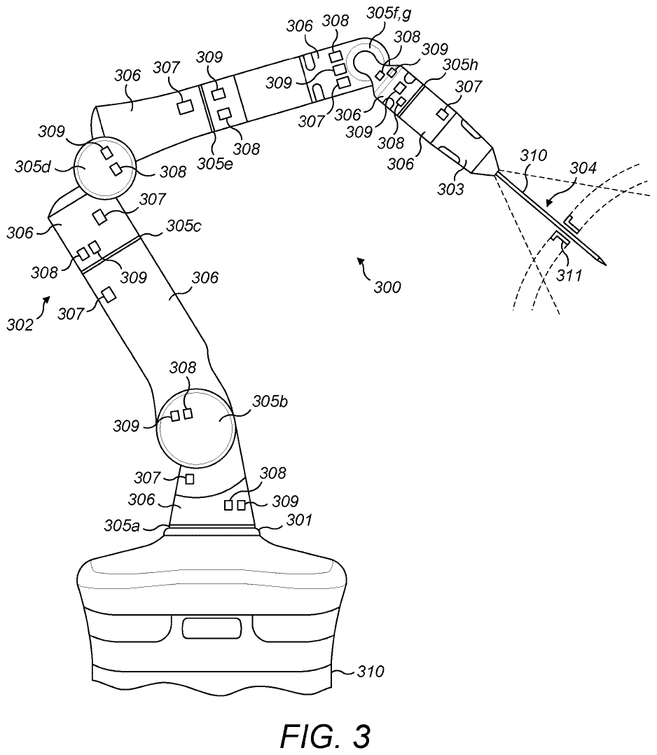

FIG. 3 shows an example of a surgical robot 300. The robot comprises a base 301 which is fixed in place when a surgical procedure is being performed. Suitably, the base 301 is mounted to a support structure. In FIG. 3, the support structure is a cart 310. This cart may be a bedside cart for mounting the robot at bed height. Alternatively, the support structure may be a ceiling mounted device, or a bed mounted device.

A robot arm 302 extends from the base 301 of the robot to a terminal end 303 for attaching to a surgical instrument 304. The robot arms 201a, 201b shown in FIG. 2 may have the same features as the robot arm 302 shown in FIG. 3. The arm is flexible. It is articulated by means of multiple flexible joints 305 along its length. In between the joints are rigid arm links 306. Suitably, the joints are revolute joints. The robot arm has at least seven joints between the base and the terminal end. The robot arm 300 illustrated in FIG. 3 has eight joints in total between the base 301 and the terminal end 303. The joints include one or more roll joints (which have an axis of rotation along the longitudinal direction of the arm links on either side of the joint), one or more pitch joints (which have an axis of rotation transverse to the longitudinal direction of the preceding arm link), and one or more yaw joints (which also have an axis of rotation transverse to the longitudinal direction of the preceding arm link and also transverse to the rotation axis of a co-located pitch joint). In the example of FIG. 3: joints 305a, 305c, 305e and 305h are roll joints; joints 305b, 305d and 305f are pitch joints; and joint 305g is a yaw joint. The order of the joints sequentially from the base 301 of the robot arm to the terminal end 303 of the robot arm is: roll, pitch, roll, pitch, roll, pitch, yaw, roll. There are no intervening joints in FIG. 3.

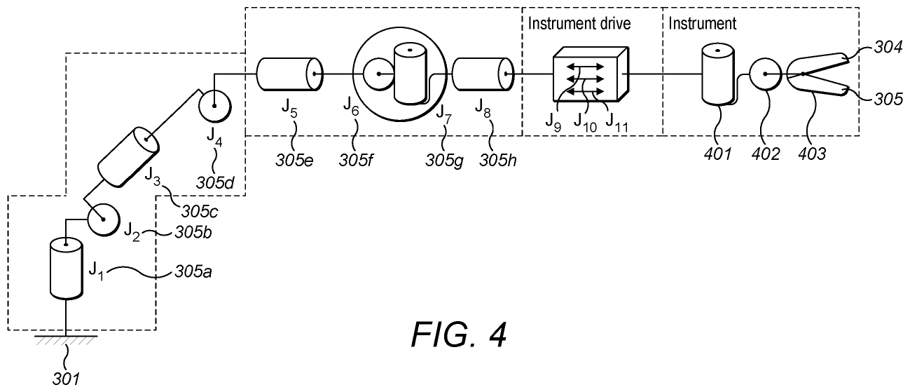

The joints of the surgical robot arm of FIG. 3 are illustrated on FIG. 4. The robot arm is articulated by eight joints. Roll joint J₁ 305a is adjacent to the base 301, and is followed by a pitch joint J₂ 305b. The pitch joint J₂ has a rotation axis perpendicular to the rotation axis of the roll joint J₁. Roll joint J₃ 305c is adjacent to the pitch joint J₂, and is followed by a pitch joint J₄ 305d. The pitch joint J₄ has a rotation axis perpendicular to the rotation axis of the roll joint J₃. Roll joint J₅ 305e is adjacent to the pitch joint J₄, and is followed by a pitch joint J₆ 305f and a yaw joint J₇ 305g, followed by a roll joint J₈ 305h. The pitch joint J₆ and yaw joint J₇ form a compound joint, which may be a spherical joint. The pitch joint J₆ and the yaw joint J₇ have intersecting axes of rotation.

The end of the robot arm distal to the base can be articulated relative to the base by movement of one or more of the joints of the arm. The rotation axes of the set of distal joints J₅, J₆, J₇ and J₈ all intersect at a point on the surgical robot arm. Reference is made to a wrist. Suitably, the wrist is a portion of the robot arm which rigidly couples to the proximal end of an instrument when that instrument is attached to the robot arm. The wrist has a position and an orientation. For example, the position of the wrist may be the intersection of the rotation axes of J₅, J₆, J₇ and J₈. Alternatively, the position of the wrist may be the intersection of one or more rotation axes of joints of the instrument. Alternatively, the position of the wrist may be the intersection of one or more rotation axes of the distal joints of the robot arm and one or more rotation axes of joints of the instrument. The surgical robot arm illustrated in FIGS. 3 and 4 has a redundant joint. For a given position of the wrist relative to the base of the surgical robot arm, there is more than one configuration of the joints J₁ to J₄. Thus, the surgical robot arm can adopt different poses whilst maintaining the same wrist position.

The surgical robot arm could be jointed differently to that illustrated in FIGS. 3 and 4. For example, the arm may have fewer than eight or more than eight joints. The arm may include joints that permit motion other than rotation between respective sides of the joint, for example a telescopic joint.

Returning to FIG. 3, the surgical robot arm comprises a set of motors 307. Each motor 307 drives one or more of the joints 305. Each motor 307 is controlled by a joint controller. The joint controller may be co-located with the motor 307. A joint controller may control one or more of the motors 307. The robot arm comprises a series of sensors 308, 309. These sensors comprise, for each joint, a position sensor 308 for sensing the position of the joint, and a torque sensor 309 for sensing the applied torque about the joint's rotation axis. The torque applied about a joint's rotation axis includes any one or combination of the following components: torque due to gravity acting on the joint, torque due to inertia, and torque due to an external force applied to the joint. One or both of the position and torque sensors for a joint may be integrated with the motor for that joint. The outputs of the sensors are passed to the control system.

The surgical instrument 304 attaches to a drive assembly at the terminal end of the robot arm 303. This attachment point is at all times external to the patient. The surgical instrument 304 has an elongate profile, with a shaft spanning between its proximal end which attaches to the robot arm and its distal end which accesses the surgical site within the patient's body. The surgical instrument may be configured to extend linearly parallel with the rotation axis of the joint 305h of the arm. For example, the surgical instrument may extend along an axis coincident with the rotation axis of the joint 305h of the arm.

The proximal end of the surgical instrument and the instrument shaft may be rigid with respect to each other and rigid with respect to the distal end of the robot arm when attached to it. An incision is made into the patient's body, through which a port is inserted. The surgical instrument may penetrate the patient's body through the port to access the surgical site. Alternatively, the surgical instrument may penetrate the body through a natural orifice of the body to access the surgical site. At the proximal end of the instrument, the shaft is connected to an instrument interface. The instrument interface engages with the drive assembly at the distal end of the robot arm. Specifically, individual instrument interface elements of the instrument interface each engage a respective individual drive assembly interface element of the drive assembly. The instrument interface is releasably engageable with the drive assembly. The instrument can be detached from the robot arm manually without requiring any tools. This enables the instrument to be detached from the drive assembly quickly and another instrument attached during an operation.

At the distal end of the surgical instrument, the distal end of the instrument shaft is connected to an end effector by an articulated coupling. The end effector engages in a surgical procedure at the surgical site. The end effector may be, for example, a pair of jaws, a pair of monopolar scissors, a monopolar hook, a bipolar instrument, a needle holder, a fenestrated grasper, or a scalpel. The articulated coupling comprises several joints. These joints enable the pose of the end effector to be altered relative to the direction of the instrument shaft. The end effector itself may also comprise joints. The end effector illustrated in FIGS. 3 and 4 has a pair of opposing end effector elements 304, 305. The joints of the end effector are illustrated on FIG. 4 as a pitch joint 401, a yaw joint 402 and a pinch joint 403. The pitch joint 401 is adjacent to the shaft of the instrument and rotates about an axis perpendicular to the longitudinal axis of the instrument shaft. The yaw joint 402 has a rotation axis perpendicular to the rotation axis of the pitch joint 401. The pinch joint 403 determines the spread of the end effector elements. In practice, the pinch joint 403 may be another yaw joint which has the same rotation axis as the yaw joint 402. Independent operation of the two yaw joints 402, 403 can cause the end effector elements to yaw in unison, and/or to open and close with respect to each other.

Drive is transmitted from the robot arm to the end effector in any suitable manner. For example, the joints of the instrument may be driven by driving elements such as cables, push rods or push/pull rods. These driving elements engage the instrument interface at the proximal end of the instrument. The drive assembly at the terminal end of the robot arm comprises instrument drive joints which transfer drive from the surgical robot arm to the instrument interface via the respective interface elements described above, and thereby to the instrument joints. These instrument drive joints are shown on FIG. 4 as joints J₉, J₁₀ and J₁₁. FIG. 4 illustrates three instrument drive joints, each one of which drives one of the three joints of the instrument.

Suitably, the instrument drive joints are the only means by which drive is transferred to the instrument joints. The robot arm may have more or fewer than three instrument drive joints. The surgical instrument may have more or fewer than three joints. The instrument drive joints may have a one-to-one mapping to the instrument joints that they drive, as shown in FIG. 4. Alternatively, an instrument drive joint may drive more than one instrument joint.

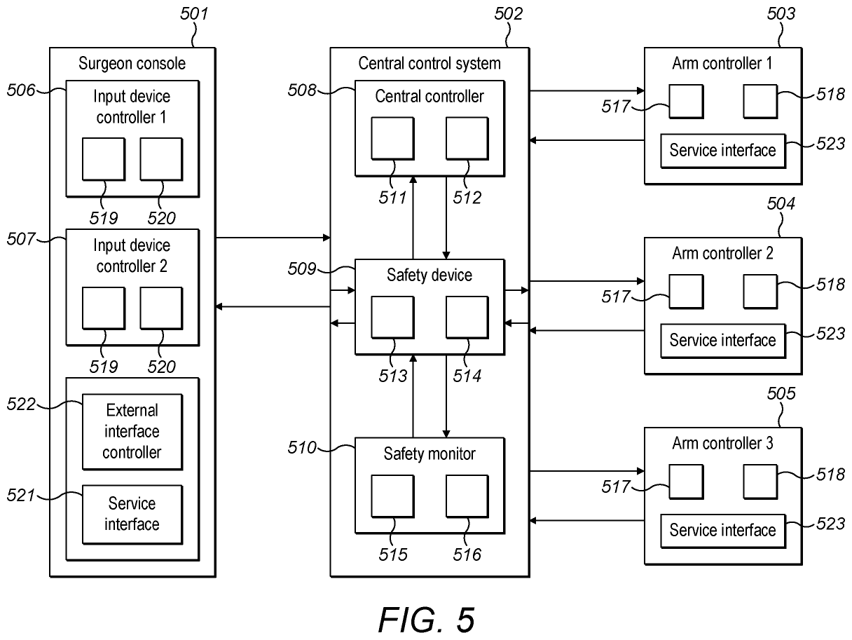

A control system connects the surgeon console to the one or more surgical robots. Such a control system is illustrated in FIG. 5. The surgeon console 501 is connected by a bi-directional communications link to a central control system 502. Each surgeon input device is controlled by an input device controller 506, 507. Each input device controller 506, 507 is communicatively coupled to the central control system 502. The central control system 502 is connected by a bi-directional communications link to an arm controller 503, 504, 505 of each surgical robot arm of the surgical robotic system. Each arm controller is co-located with a surgical robot arm. The arm controller may be located in the surgical robot arm. Alternatively, the arm controller may be located in the support structure which supports the surgical robot arm, for example in the cart of the arm.

The central control system is remotely located from at least one of the surgical robot arms. Suitably, the central control system is remotely located from all the surgical robot arms in the surgical robotic system. The central control system may be located at the surgeon console. Alternatively, the central control system may be co-located with one of the arm controllers. The central control system may be located remote from both the surgeon console and all the arm controllers. The central control system may be distributed between two or more of: (i) the surgeon console, (ii) the robot arms, (ii) the support structure of the robot arms, and (iv) a location remote from (i) to (iii).

The central control system 502 comprises a central controller 508, a safety device 509 and, optionally, a safety monitor 510. The central controller 508 is configured to: receive communications from the surgeon console 501 identifying the surgeon input device inputs, and communications from the surgical robot comprising surgical robot state data such as sensed joint positions and sensed joint torques. As described in more detail below, in response to these inputs, the central controller 508 generates control signals which it sends to the arm controller of the surgical robot being controlled. The central controller 508 may have additional functionality. For example, the central controller 508 may be configured to provide and/or control at least part of a graphical user interface provided to the surgeon for providing input. The central controller 508 comprises a processor 511 and a memory 512. The memory 512 stores, in a non-transient way, software code that can be executed by the processor 511 to cause the processor to control the surgeon console and the one or more surgical robot arms and instruments in the manner described herein.

The central control system 502 may also comprise a safety device 509, and, optionally, a safety monitor 510. The safety device 509 is a hardware device situated between the central controller 508 and the other robot arms and surgeon console. Communications to and from the central controller 508 pass through the safety device 509. Since the communications to and from the central controller 508 pass though the safety device 509, the safety device 509 can control the communications to and from the central controller 508. Specifically, the safety device 509 can prevent communications between the central controller 508 and a robot arm or the surgeon console when it has been detected that the surgical robot system is in a fault state.

The safety device 509 may be operable to selectively filter the communications to and/or from the central controller 508 based on one or more filter criteria. In some cases, the safety device 509 may comprise one or more programmable filters which can be programmed or configured to filter certain communications to and/or from the central controller 508. The safety device 509 comprises a processor 513 and a memory 514. The memory 514 stores, in a non-transient way, software code that can be executed by the processor 513 to cause the processor to operate as described above.

The central control system 502 may also comprise a safety monitor 510. The safety monitor 510 is configured to independently verify the operation of the central controller 508, and/or one or more other components and devices in the system, by monitoring the communications to and from the central controller 508. In these cases, the safety device 509 may be configured to send a copy of at least a portion, of the communications to and/or from the central controller 508 to the safety monitor 510. The safety monitor 510 is then configured to analyse the received communications to determine if the surgical robot system is in a fault state. If the safety monitor 510 detects that the surgical robot system is in a fault state, the safety monitor 510 may be configured to cause the safety device 509 to filter at least a portion of the communications to and/or from the central controller 508. Thus, the safety monitor 510 may prevent faulty communications from reaching the central controller 508. Similarly, the safety monitor 510 may prevent faulty communications from being sent from the central controller 508. The safety monitor 510 comprises a processor 515 and a memory 516. The memory 516 stores, in a non-transient way, software code that can be executed by the processor 515 to cause the processor to operate as described above.

Each of the arm controllers comprises a processor 517 and a memory 518. The memory 518 stores, in a non-transient way, software code that can be executed by the processor 517 to cause the processor to control the surgeon console and the one or more surgical robot arms and instruments in the manner described herein.

Each of the input device controllers comprises a processor 519 and a memory 520. The memory 520 stores, in a non-transient way, software code that can be executed by the processor 519 to cause the processor to control the surgeon input device and/or one or more surgical robot arms and instruments in the manner described herein.

The communications links between the central control system 502 and the surgeon console 501 and robot arms controllers 503, 504, 505 may be any suitable communications links that enables data communications between the central control system 502 and the component. Examples of suitable communications links include, but are not limited to, a wired communications link (e.g. an Ethernet, Token Ring, or RS232 link), or a wireless communications link (e.g. a Wi-Fi, Bluetooth, Bluetooth LE, or NFC link).

The surgeon manipulates the input device, for example by translating it, rotating it, operating a button, operating a switch, operating a trigger, operating a thumb stick, operating a touch pad, or operating a roller. The input device controller of the input device translates the surgeon's input to the input device to commands which it sends to the central control system 502. The central control system 502 receives commands from the input device controller(s). The commands from the input device controller indicate a change in the desired position and/or pose of a distal end of a surgical instrument. The control system converts the commands received from the input device controller to drive signals. This conversion is carried out by one or a combination of the central controller and the surgical robot arm controller of the surgical robot arm associated with the surgeon input device/input device controller. The robot arm controller sends the drive signals to the joint controllers of the surgical robot arm and/or surgical instrument associated with the surgeon input device/input device controller. Those joint controllers respond by driving the joint motors accordingly. The joints are thereby driven to cause the end effector to adopt the desired position and/or pose commanded by the surgeon input device. Manipulation of the surgical instrument is thereby controlled by the control system in response to manipulation of the surgeon input device.

The control system receives inputs from the position and torque sensors on the joints of the surgical robot arms. The control system determines the current configuration of a surgical robot arm using the known sequence of joints and links in the arm, and the sensed joint positions. From the current configuration of the surgical robot arm and the attached surgical instrument, and the known mass and dimensions of the links and joints of the robot arm and instrument, the control system determines the torque due to gravity acting on each joint. The control system sends gravity compensating drive signals to the joint controllers of the robot arm. The joint controllers respond by driving the joint motors so as to counteract the force of gravity acting on each joint. In other words, each joint motor applies a torque which exactly opposes the calculated gravitational force acting on the joint. In the absence of commands from the surgeon input device and/or external forces (other than gravity) acting on the robot arm, the robot arm is thereby held in position against gravity. It does not droop under the force of gravity. In practice, each drive signal sent by the control system to a joint controller for driving a joint motor may be resolved into a component which drives the joint in accordance with the input received from the surgeon input device, and a component which counteracts gravity. In some modes, as discussed below, the drive signal may also comprise a component which drives the joint to conform with an external force applied to the robot arm.

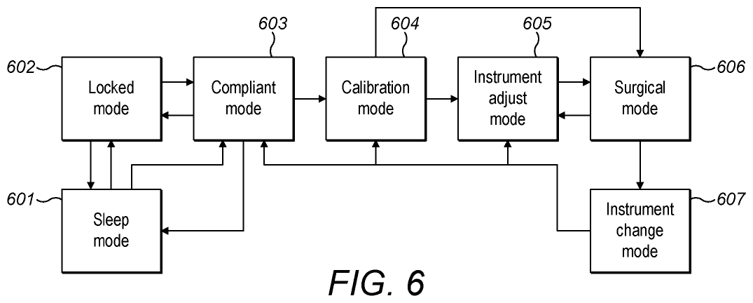

A surgical robot arm is operable in a clinical mode within a surgical setting. In other words, in an operating theatre in which the surgical robot is to be used for performing surgery in response to inputs received at a surgeon input device of the surgeon console, the surgical robot arm is placed in a clinical mode. Within the clinical mode, the surgical robot arm is operable in a number of different sub-modes of the clinical mode, referred to herein after as operating modes. FIG. 6 illustrates some exemplary operating modes of the surgical robot arm, and the transitions permitted between those operating modes.

The surgical robot arm is operable in a sleep mode 601. In the sleep mode 601, the robot arm may adopt a configuration suitable for storing or transporting the robot arm. Such a configuration may be a compact configuration. For example, in this compact configuration the arm links on either side of the joint 305d may be substantially parallel to one another. Suitably, a surgical instrument 304 is not attached to the robot arm when it is operating in the sleep mode 601.

In the sleep mode 601 the robot arm may resist external forces so as to maintain its configuration. Brakes may be applied at one or more of the joints 305a-g of the robot arm such that the robot arm can resist external forces so as to maintain its configuration. These brakes may be of any suitable type, such as electronic, magnetic or mechanical brakes. In the sleep mode 601, the robot arm does not change its configuration in response to inputs at the remote surgeon console.

The robot arm may be operable in a locked mode 602. In the locked mode, the robot arm may adopt a “horse-shoe” configuration (e.g. of the type shown in FIG. 3). The “horse-shoe” configuration is preferable in the locked mode 602 as the orientation of the joints of the robot arm are away from their joint limits and singular configurations. That is, the “horse-shoe” configuration is such that a large range of movement of the robot arm is possible from that configuration. In the “horse-shoe” the robot arm can be placed near the port in a configuration which is similar to the final/optimal configuration that will be used when performing surgery—so that the operating staff have a good idea on how the surgical setup will look. On transitioning from the sleep mode 601 to the locked mode 602, the configuration of the robot arm may be altered from a compact configuration to the “horse-shoe” configuration. Such a change may be driven by one or more of the series of motors 307a-h.

A surgical instrument 304 may be attached to the robot arm when it is operating in the locked mode 602. In the locked mode 602 the robot arm may resist external forces so as to maintain its configuration. Brakes may be applied at one or more of the joints 305a-g of the robot arm such that the robot arm can resist external forces so as to maintain its configuration. Said brakes may be of any suitable type, such as electronic, magnetic or mechanical brakes. In the locked mode 602, the robot arm does not change its configuration in response to inputs at the remote surgeon console.

The operating mode of the robot arm may also be transitioned from the locked mode 602 to the sleep mode 601, such as when the robot arm is being prepared for storage after a procedure has been completed.

The robot arm may be operable in a compliant mode 603. As shown in FIG. 6, the operating mode of the robot arm may be caused to transition from the locked mode 602 to the compliant mode 603. The operating mode of the robot arm may also be caused to transition directly from the sleep mode 601 to the compliant mode 603.

In the compliant mode 603, the configuration of the robot arm is changeable in response to external forces applied directly to that robot arm. For example, a member of the bedside team (e.g. an operating room nurse) may apply forces directly to a robot arm (e.g. by pushing a joint of the robot arm). In the compliant mode 603 the control system controls the robot arm to maintain a position in which it is placed by means of external forces applied directly to the robot arm.

To achieve this, the control system receives inputs from the position and force sensors 308 and 309. From the position sensors the control system can determine the current configuration of the robot arm. The control system stores for each element of the robot arm, and the surgical instrument, its mass, the distance of its centre of mass from the preceding joint of the robot arm and the relationship between the centre of mass and the positional output of the position sensor for the preceding joint. The current configuration of the robot arm could be inferred by other means. For example, camera-based positioning systems may be used to track points in space, such as fiducial markers attached to the robot arm. This technique could be used to determine the joint angles. Other techniques include inferring the position of a joint using current sensors. For example, the position of a joint can be inferred from the amount of current passing through the motor and assuming a given relationship to be constant.

Using that information, the control system models the effect of gravity on the components of the robot arm for the current configuration of the robot arm and estimates a force (e.g. a torque) due to gravity on each joint of the robot arm. The processor then drives the motor 307 of each joint to apply a force (e.g. a torque) that will exactly oppose the calculated gravitational force. With this control strategy an operator (e.g. an operating room nurse) can push or pull any part of the robot arm to a desired position, and the part will stay in that position notwithstanding the effect of gravity on it and on any parts depending from it. A force on the arm may result in a torque about multiple joints. The control system can be programmed to decide to prioritise certain ones of the joints for neutralising the torque. In examples, some joints could be locked in position and others could move compliantly, the position of a given link or point in space could be prioritized rather than sets of joints.

Each motor 307 may be controlled in response to the force (e.g. torque) measured about the respective joint. When the measured force at a joint is adjusted for gravity, any remaining sensed force represents a force applied by an external force (e.g. due to a push or pull on the robot arm). In response to that force the control system may control the respective motor 307 so as to alter the configuration of the robot arm. For example, this may be achieved by controlling the motors 307 to move their respective joints 305 in a direction so as to reduce the measured force, and at a rate dependant on the magnitude of the measured force. In this way, the member of the bedside staff may feel that that the robot arm is moving freely in response to the force they are applying—when in fact it is the motors of the robot arm driving the movement.

In the compliant mode 603, the control system does not convert detected manipulation of the surgeon input device(s) to drive signals for moving joints of the robot arm. Any inputs the control system receives from the surgeon input device are not converted to movement of the robot arm.

The compliant mode 603 can be used to insert the surgical instrument into a port in the patient's body. With the robot arm in the compliant mode 603, an operator (e.g. an operating room nurse) can grasp one or both of the robot arm 302 and the surgical instrument 304. The operator can then apply external forces so as to alter the configuration of the robot arm 302 such that the elongate axis of the shaft of the instrument is roughly aligned with the passageway through the port. The operator can then apply an external force (e.g. push) to the robot arm and/or the instrument such that the instrument moves roughly parallel to its elongate axis and passes into the passageway in the port.

The operating mode of the robot arm may also be transitioned from the compliant mode 603 to the locked mode 602 or the sleep mode 601, such as when the robot arm is being prepared for storage after a procedure has been completed.

The robot arm may be operable in a calibration mode 604. As shown in FIG. 6, the operating mode of the robot arm is caused to transition from the compliant mode 603 to the calibration mode 604.

In the calibration mode 604, the surgical robot arm is driven to conform to external forces applied to the robot arm as described above with respect to the compliant mode 603. The surgical instrument may be inserted into the patient in either the compliant mode or the calibration mode.

As with the compliant mode, in the calibration mode, the control system does not convert detected manipulation of the surgeon input device(s) to drive signals for moving joints of the robot arm. Any inputs the control system receives from the surgeon input device are not converted to movement of the robot arm.

During setup of the surgical robotic system, the calibration mode is used to determine a fulcrum or virtual pivot point. The virtual pivot point is the natural centre of rotation of an instrument having a rigid shaft as that instrument moves in the patient's body. The virtual pivot point is a fulcrum about which the surgical instrument pivots when the configuration of the surgical robot arm is altered whilst inside the port in the patient's body. A port is inserted into the abdominal wall of the patient. The port is of the order of 2-10 cm long. The instrument is inserted into the patient's body through the port. The virtual pivot point lies along the length of the port. The exact location of the virtual pivot point depends on the patient's anatomy, and hence differs from patient to patient.

The virtual pivot point can be determined using the following method. With the instrument located in the port, an operator moves the distal end of the robot arm in directions generally transverse to the instrument shaft 310. This motion causes the port 311 to exert a lateral force on the instrument shaft where it passes through the port, with the result that the instrument applies a torque to the joints of the arm—in this case joints J₆ 305f and J₇ 305g—whose axes are transverse to the longitudinal axis of the instrument shaft. The position of each arm joint is measured by its associated position sensor 308, and this sensed position is output to the control system. The torque at each arm joint is measured by its associated torque sensor 309, and this sensed torque is output to the control system. Thus, as the operator moves the distal end of the robot arm laterally the control system receives sensed inputs indicating the position and forces on the arm joints. That information allows the control system to estimate: (a) the position of the distal end of the robot relative to the fixed base and (b) the vector of the instrument shaft relative to the distal end of the robot. Since the instrument shaft passes through the passageway of the port, the passageway of the port must lie along that vector. As the distal end of the robot arm is moved, the controller calculates multiple pairs of distal end positions and instrument shaft vectors. Those vectors all converge, from their respective distal end position, on the natural rotation centre of the passageway of the port 311, i.e. at the location of the virtual pivot point in the passageway of the port. By collecting a series of those data pairs and then solving for the mean location where the instrument shaft vectors converge, the control system determines the virtual pivot point relative to the base. The control system then stores the determined fulcrum/virtual pivot point in memory for later use.

Once the virtual pivot point is determined in the calibration mode 604, it is set for the remainder of the operating modes illustrated on FIG. 6. The virtual pivot point is stored by the control system. In each of the other modes illustrated in FIG. 6, the surgical robot arm is always driven such that the longitudinal axis of the shaft of the attached surgical instrument intersects the virtual pivot point (whether or not the instrument is actually attached to the robot arm). The longitudinal axis of the shaft of the surgical instrument has a known relationship to the longitudinal axis of the terminal end of the robot arm. For example, the longitudinal axis of the shaft of the surgical instrument may be coincident with the longitudinal axis of the terminal end of the robot arm.

After determining the fulcrum, the robot arm may be operable in an instrument adjust mode 605. As shown in FIG. 6, the operating mode of the robot arm may be caused to transition from the calibration mode 604 to the instrument adjust mode 605. The robot arm may also be caused to transition from the calibration mode 604 directly to the surgical mode 606. The operating mode of the robot arm may be caused to transition from the surgical mode 606 to the instrument adjust mode 605.

In the instrument adjust mode 605, the control system drives the surgical robot arm to conform to external forces applied to the robot arm (as described above with respect to the calibration mode), whilst retaining the intersection of the longitudinal axis of the shaft of the surgical instrument with the virtual pivot point determined in the calibration mode. The instrument adjust mode 605 can be used to adjust the position of the instrument within the patient's body. For example, the instrument adjust mode 605 may be used to enable a member of the bedside team to push the instrument into the patient's body such that the end effector reaches the surgical site, following setting of the virtual pivot point in the calibration mode. In the instrument adjust mode 605, the control system drives the joints of the robot arm to compensate for gravity (as described above). In the instrument adjust mode 605, the control system does not convert any manipulation of the surgeon input device to drive signals for driving the joints of the robot arm.

From the instrument adjust mode 605, the surgical robot arm can transition to the surgical mode 606. In the surgical mode 606, the control system responds to inputs received from a surgeon input device by converting those inputs to control signals for controlling the motion of the surgical robot arm and/or surgical instrument associated with that surgeon input device (as described above). The end effector of the surgical instrument thereby moves as commanded by the surgeon input device. When performing the conversion, the control system maintains an intersection between the longitudinal axis of the shaft of the surgical instrument and the virtual pivot point.

The surgical mode 606 may be a semi-compliant mode. In other words, the robot arm may exhibit some compliant behaviour towards external forces applied to the robot arm. For example, in the surgical mode 606, the control system may respond to a sensed external force applied proximal to the elbow joint 305d by controlling the motors driving the elbow joint 305d and the surrounding joints of the arm to drive those joints to comply with that sensed external force. In this way, a member of the bedside team can push the elbow joint 305d or a part of the arm proximal to the elbow joint out of the way to enable them to access the patient during the surgical mode. In order to implement this, the control system may define a permitted area/volume for one or more parts of the robot arm that are designated as compliant such that movement of those parts in response to externally applied forces is confined within the permitted area/volume. The permitted area/volume is defined such that movements within that area/volume in response to externally applied forces do not cause the configuration of the instrument to be affected. The robot arm is only semi-compliant in the surgical mode 606 because the control system does not respond by conforming to an external force applied to any part of the robot arm other than the parts designated as compliant.

From the surgical mode 606, the surgical robot arm can transition to the instrument change mode 607. The instrument change mode 607 is engaged in order to remove and/or insert the instrument from/into the patient's body. In the instrument change mode 607, the control system drives the surgical robot arm to conform to the component of a sensed external force applied to the robot arm along the longitudinal axis of the surgical instrument towards or away from the surgical robot arm. Whilst in the instrument change mode 607, the control system retains the intersection of the longitudinal axis of the shaft of the surgical instrument with the virtual pivot point determined in the calibration mode. The control system conforms to the sensed external force in the same manner as described above with respect to the calibration mode, the only differences being that (i) the control system only conforms to the component of the sensed force in the specified directions (i.e. along the longitudinal axis of the surgical instrument towards or away from the surgical robot arm), and (ii) the control system maintains intersection of the surgical instrument with the virtual pivot point.

At the end of an operation when surgical instruments are being removed from the surgical site, or mid-operation when a surgical instrument is being exchanged for another one, a member of the bedside team uses the instrument change mode 607 to enable them to pull the instrument out of the patient's body, and then insert another instrument into the patient's body. In the instrument change mode 607, the control system prevents force applied by the member of the bedside team in any direction other than the longitudinal axis of the surgical instrument towards or away from the surgical site from being converted to corresponding movement of the surgical robot arm. Thus, no lateral force applied by the member of the bedside team is converted to corresponding movement of the surgical robot arm. This ensures that extraction of the surgical instrument is along the line of entry between the port and the surgical site, thus avoiding damage to tissue away from this line.

In the instrument change mode 607, the control system limits the conversion of force applied by the member of the bedside team along the longitudinal axis of the surgical instrument towards the patient's body to corresponding movement of the surgical robot arm. This limit is such that the end effector of the attached instrument cannot advance further into the patient's body than the end effector of the instrument at the time that the instrument change mode was entered. This limit applies to the same instrument that was attached to the arm at the time that the instrument change mode was entered during instrument extraction. This limit also applies to the newly attached instrument which is inserted into the patient's body following instrument change. This ensures that the surgical instrument cannot be pushed further into the patient's body causing damage to the surgical site.

In the instrument change mode 607, the control system drives the joints of the robot arm to compensate for gravity (as described above). In the instrument change mode 607, the control system does not convert any manipulation of the surgeon input device to drive signals for driving the joints of the robot arm.

The surgical robot can transition from the instrument change mode 607 to any of the compliant mode 603, calibration mode 604, or instrument adjust mode 605.

Each robot arm of the system shown in FIG. 2 can be in any of the operating modes described with respect to FIG. 6 at any time, regardless of the operating mode any other robot arm is in. However, in order for a surgeon to have operative control of a surgical instrument from a surgeon input device in surgical mode, an endoscope may be required to be connected to another robot arm and have been successfully calibrated in the calibration mode.

From time to time support engineers access the surgical robotic system to run tests and diagnostics, for example for the purpose of identifying and recovering faulty components, or development testing. They also access the surgical robotic system in order to perform software upgrades. In order to perform these tasks, the component(s) of the surgical robotic system which needs to be accessed is put into a service mode. In this service mode, the component of the surgical robotic system can be controlled by the service engineer via a service interface. For example, the service engineer may connect a laptop to the component via a service interface and control the functionality of the component from there. The components include, for example, the arm controllers 503, 504, 505, the surgeon input device controllers 506, 507, the central controller 508, the safety device 509, and the safety monitor 510. A service interface 521 of the surgeon console is illustrated on FIG. 5, along with an external interface controller 522 for controlling it. Similarly, service interfaces 523 connected to each arm controller are illustrated on FIG. 5.

Each component of the surgical robotic system may initially power up in the clinical mode. To enter the service mode on a component, the service engineer may connect a servicing device to the service interface, and then send a request to enter the service mode from the servicing device to the component. A handshake sequence between the servicing device and the component may then be performed to link them together and cause the component to transition to the service mode. That handshake sequence may be based on exchange of a secret key, that secret key being stored locally on each of the component and servicing device. For example, the component may send a challenge to the servicing device in response to the received request to enter the service mode. That challenge may consist of, for example, a random number. The servicing device receives the challenge and generates a hash key by performing a hash function on a combination of the received challenge and the stored secret key. The servicing device sends the hash key to the component. The component also generates a hask key by performing a hash function on a combination of the challenge and its stored secret key. The component compares its locally generated hash key to the hash key received from the servicing device. If the hash keys match, then the handshake sequence is deemed successful by the component, and it responds by transitioning to the service mode.

The servicing device may connect to each component of the surgical robotic system to be put in the service mode individually, to hence cause that component to transition to the service mode independently of the other components. Thus, one component may be in the service mode whilst other components are in the clinical mode. In this scenario, a component that is in the clinical mode may not be informed that the other component is in the service mode. For example, the arm controller 1 503 may not be informed that the central controller 508 or a surgeon input device controller 1 506 is in the service mode. One or more components that are in the clinical mode may be informed that another component is in the service mode. For example, the central controller 508 and/or the safety monitor 510 may be informed that one of the surgeon input device controllers 506, 507 or component of the central control system 502 is in the service mode. The servicing device may command the component in the service mode to send a message to the central controller and/or the safety monitor identifying that that component is in the service mode.

Alternatively, the servicing device may connect centrally to the surgical robotic system, for example to central controller 508, and cause the surgical robotic system as a whole to transition to the service mode.

The functionality of the surgical robotic system in the service mode generally mirrors that of the clinical mode described above, except in the following ways.

In the service mode, changes are able to be made to the configuration and/or state and/or parameters of the part of the surgical robotic system in the service mode. For example, control parameters of the surgical robot arm can be changed. An example control parameter that can be changed is the gains used in the control algorithms for moving the robot arm and/or instrument. A higher gain causes the robot arm and/or instrument to respond more quickly to inputs from the surgeon input device. A lower gain causes the robot arm and/or instrument to respond slower to inputs from the surgeon input device. Other example control parameters that can be changed are the torque sensor settings. For example, the threshold torque at which the control system responds to externally detected force in a compliant manner may be altered. Another example of a control parameter that can be changed is the status of an arm or the mode that the arm is in. In the service mode, changes may be applied to one, a plurality or all of the operating modes described with reference to FIG. 6.

To enable the engineer to utilise the service mode unimpeded, several safety features of the clinical mode are disabled in the service mode. For example, the fault check on the communication link between the arm controllers 503, 504, 505 and the joint controllers of the robot arm may be disabled to allow the engineer to perform testing in scenarios which would cause a fault to be determined in the clinical mode, for example if those communications are at a lower rate than a threshold, or the communication link is to be reset or turned off for reprogramming. As another example, a safety check to determine if the service mode has been entered is disabled in the service mode. This enables code which is not accessible in the clinical mode to be run in the service mode. This code enables the service engineer to carry out tests and change parameters which are not available actions in the clinical mode. The safety features which are disabled may be ones which are implemented by the safety device 509 or safety monitor 510. Alternatively, they may be safety checks implemented at any of the other controllers in the control system shown in FIG. 5.

The surgical robotic system in the service mode is typically not granted regulatory approval to be used to perform surgery. The surgical robotic system must be transitioned to the clinical mode in order to be used to perform surgery within the permissions granted by the regulatory bodies. This is carried out by power cycling each component of the surgical robotic system which is in the service mode. Each component, when power cycled, reboots in the clinical mode. When the component boots up in the clinical mode, it returns the configuration and parameters of the component to the predetermined states of the clinical mode. When power cycled, the component performs a Power-On-Self-Test (POST) sequence which is a series of tests to check it is operating within required parameters. Upon successful completion of the POST sequence, the component may be transitioned to a state in which it can be prepared for surgery. For example, upon successful completion of the POST sequence the surgical robot arm can be transitioned from the sleep mode 601 to the locked mode 602 or compliant mode 603. When the component reboots in clinical mode, the safety checks which had been disabled in the service mode are re-enabled. Thus, the component rebooted in clinical mode is safe for use to perform surgery.

In order to ensure that the surgical robotic system is not used for performing surgery when any component of the surgical robotic system is in service mode, the following safety mechanisms are employed by the control system in the service mode.

Firstly, a surgical robot arm is prevented from being transitioned into the calibration mode 604 if the surgical robot arm is in service mode. The calibration mode must be completed and the virtual pivot point/fulcrum determined in order to transition to the surgical mode 606, thus preventing the surgical robot arm from being transitioned into the calibration mode 604 prevents the surgical robot arm from being used to perform surgery. The surgical robot arm may also be prevented from being transitioned into the surgical mode 606 if the surgical robot arm is in service mode. This prevents the surgical robot arm from being used to perform surgery in the service mode.

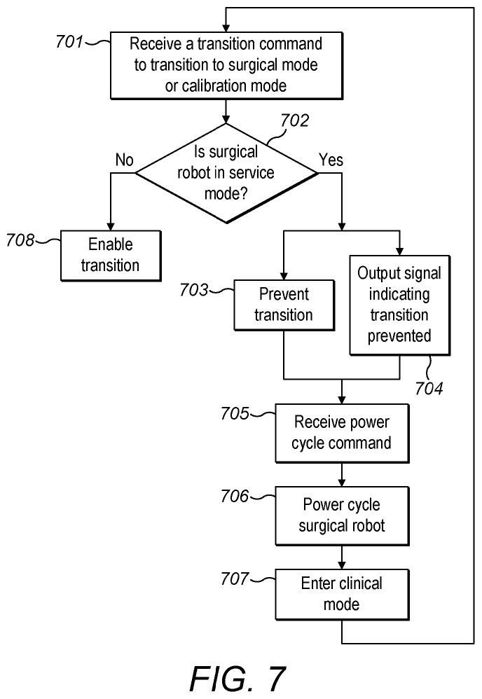

FIG. 7 illustrates an exemplary flowchart for implementing the steps of a safety mechanism to prevent surgery taking place in the service mode.

At step 701, the control system receives a transition command to transition the surgical robot to the surgical mode 606 or the calibration mode 604. The transition command may be received via a user input. The user input may be on the robot arm, such as a button on the robot arm. The user input may be on another part of the surgical robotic system, such as a user input on the surgeon console or a user input on the surgical cart. Suitably, the arm controller of the surgical robot arm receives the transition command. Alternatively, a different part of the control system may receive the transition command.

At step 702, the control system determines whether the surgical robot is operating in the service mode. Suitably, the arm controller performs step 702. The arm controller may store the current mode (i.e. service mode or clinical mode) of its surgical robot arm in memory 518. In this case, the arm controller retrieves the current mode from memory 518. If the control system determines that the surgical robot is operating in the service mode, then the method moves on to step 703.

At step 703, the control system prevents the transition command from being carried out. Thus, it prevents the surgical robot arm from being transitioned to the requested mode, be that the surgical mode or the calibration mode. Suitably, the arm controller performs step 703.

At step 704, the control system outputs one or more signals indicating that the transition has been prevented. For example, the arm controller may output a signal on the surgical robot indicative that the surgical robot has been prevented from transitioning to the commanded mode. This signal may be an audible signal, such as a beep. This signal may be a visual signal, such as a coloured and/or flashing light. As another example, the arm controller may message the central controller 508 that the transition has been prevented. The central controller 508 may respond to this message by outputting a signal on the surgeon console 501 indicative that the surgical robot has been prevented from transitioning to the commanded mode. This signal may be an audible signal, such as a beep. This signal may be a visual signal, such as an icon on the display which may be flashing or a coloured and/or flashing light.

In order to enable the transition to the surgical mode or the calibration mode, the surgical robot needs to be power cycled so as to cause it to reboot in clinical mode. This is illustrated in the following steps of the flowchart. At step 705, the control system receives a power cycle command to power cycle the surgical robot arm. This power cycle command may be received, for example, by the arm controller through its service interface from the servicing device. Alternatively, the power cycle command may be received from a user via a user input on the surgical robot arm or on the surgical robot arm base or support structure. For example, via an ON/OFF switch.

At step 706, in response to receiving the power cycle command, the control system power cycles the surgical robot. This may be implemented by the arm controller or the central controller 508. The power cycling may comprise performing a series of checks referred to as Power On Self-Test (POST) checks. These tests comprise, for example, checking that the motors of the joint controllers of the robot arm are powered properly, i.e. within expected parameters. Once the checks are successfully completed, then at step 707, the control system loads the surgical robot into the clinical mode. The control system applies a predetermined configuration with predefined states and parameters to the robot arm, which are those of the clinical mode.

The control method then returns to step 701. On subsequently receiving a transition command to transition the surgical robot arm to the calibration mode or surgical mode, at step 702 the arm controller this time determines that the surgical robot is not operating in the service mode, since it is operating in the clinical mode. Thus, this time, the method moves to step 708 where transitioning into the requested mode (i.e. calibration or surgical) is enabled. There may be other conditions which must be fulfilled in order for the transition to be approved by the control system. However, the control system will not prevent the transition due to the service/clinical mode status of the surgical robot.

A second safety mechanism employed by the control system in the service mode is to prevent a surgical instrument or endoscope from being engaged with a surgeon input device of the surgeon console, thereby preventing a surgeon from being able to control the instrument or endoscope using the surgeon input device of the surgeon console.

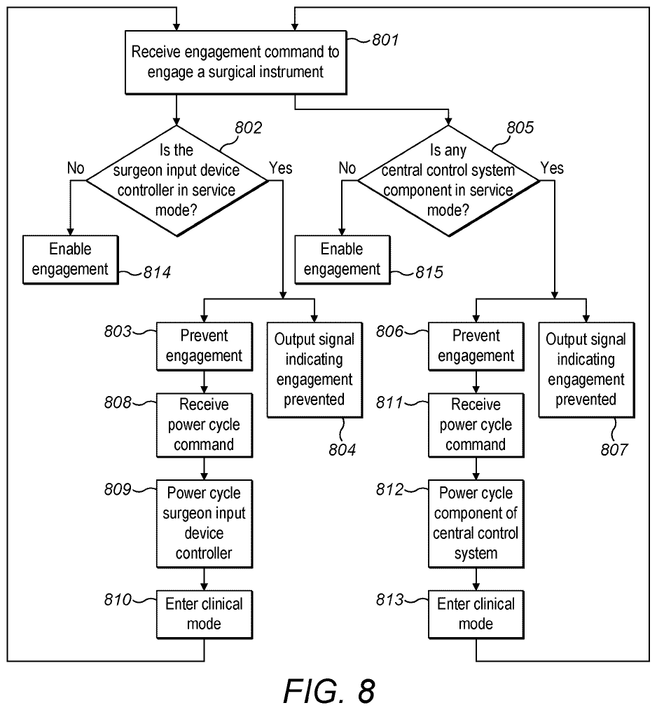

FIG. 8 illustrates an exemplary flowchart for implementing the steps of a safety mechanism to prevent the surgeon input devices engaging with a surgical instrument or endoscope in the service mode. The surgical instrument or endoscope is physically attached to the robot arm. Following this, the surgical instrument or endoscope is normally communicatively coupled with the surgeon console, and specifically with one of the surgeon input devices so that the surgical instrument/endoscope can be controlled in response to manipulation of that surgeon input device.

At step 801, the control system receives an engagement command to engage a surgical instrument or endoscope on a surgical robot arm. The engagement command may be received via a user input on the surgeon console. Typically, the surgeon specifies which surgeon input device he wants to control the instrument/endoscope via a user input on that surgeon input device. The user input may, for example, be a button or switch on the surgeon input device. Suitably, the central controller 508 receives the engagement command from the input device controller of the surgeon console. Alternatively, a different part of the control system may receive the engagement command.

At step 802, the control system determines if the surgeon input device controller is operating in the service mode. The input device controller may store the current mode (i.e. service mode or clinical mode) of its surgeon input device in memory 520. The input device controller retrieves the current mode from memory 520 and communicates it to the central controller 508. The input device controller may perform this communication once when initially powered up. The input device controller may perform this communication periodically. The central controller 508 stores the current mode of the input device controller. The central controller 508 may therefore determine whether the surgeon input device controller is in the service mode. If the surgeon input device controller is operating in the service mode, then the method moves on to step 803.

At step 803, the control system prevents the engagement command from being carried out. For example, the central controller 508 prevents the surgeon input device controller from enabling the surgeon input device to control the surgical instrument/endoscope. Thus, the surgeon is prevented from performing surgery when the surgeon input device is in service mode by being prevented from controlling the surgical instrument/endoscope with the surgeon input device of the surgeon console.

At step 804, the control system outputs one or more signals indicating that the engagement has been prevented. For example, the central controller may output a signal on the surgeon console indicative that the surgeon input device has been prevented from controlling the surgical instrument/endoscope. This signal may be an audible signal, such as a beep. This signal may be a visual signal, such as an icon on the display which may be a flashing or a coloured and/or flashing light. As another example, the central controller may message the robot arm controller that the engagement has been prevented. The robot arm controller may respond to this message by outputting a signal on the surgical robot indicative that the surgeon input device has been prevented from controlling the surgical instrument. This signal may be an audible signal, such as a beep. This signal may be a visual signal, such as a coloured and/or flashing light.

Following step 801, the control system determines if any component of the central control system is operating in the service mode in response to receiving the engagement command. Thus, the control system determines whether the central controller 508 and/or the safety device 509 and/or the safety monitor 510 is operating in the service mode. Each of these components stores its current mode (i.e. service mode or clinical mode) in memory. The safety device 509 and safety monitor 510 communicate this current mode to the central controller 508. They may do this once when initially powered up, or periodically. The central controller 508 stores the current mode of each of the components. The central controller 508 may therefore determine whether each of the central controller 508, safety device 509 or safety monitor 510 is in the service mode. If any of these components is operating in the service mode, then the method moves to step 806.

At step 806, the control system prevents the engagement command from being carried out. For example, the central controller 508 prevents the surgeon input device controller from enabling the surgeon input device to control the surgical instrument/endoscope. Thus, the surgeon is prevented from performing surgery when any component of the central control system is in service mode by being prevented from controlling the surgical instrument/endoscope with the surgeon input device of the surgeon console.

At step 807, the control system outputs one or more signals indicating that the engagement has been prevented. For example, the central controller may output a signal on the surgeon console indicative that the surgeon input device has been prevented from controlling the surgical instrument/endoscope. This signal may be an audible signal, such as a beep. This signal may be a visual signal, such as an icon on the display which may be a flashing or a coloured and/or flashing light. As another example, the central controller may message the robot arm controller that the engagement has been prevented. The robot arm controller may respond to this message by outputting a signal on the surgical robot indicative that the surgeon input device has been prevented from controlling the surgical instrument. This signal may be an audible signal, such as a beep. This signal may be a visual signal, such as a coloured and/or flashing light.

In order to enable the surgeon input device to control the surgical instrument/endoscope, the component(s) in service mode needs to be power cycled so as to cause it to reboot in clinical mode. This is illustrated in the following steps of the flowchart. At step 808/811, the control system receives a power cycle command to power cycle the component(s) in service mode. This power cycle command may be received, for example, by the surgeon console through its service interface 521 from the servicing device. Alternatively, the power cycle command may be received from a user via a user input on the surgeon console. For example, via an ON/OFF switch.

At step 809/812, in response to receiving the power cycle command, the control system power cycles the component(s) that were in service mode. This may be implemented by the central controller 508. At step 810/813, the control system loads the component(s) that were in service mode into the clinical mode. The control system applies a predetermined configuration with predefined states and parameters to the component(s), which are those of the clinical mode.

The control method then returns to step 801. On subsequently receiving an engagement command to engage a surgical instrument/endoscope with a surgeon input device, at step 802/805 the control system this time determines that neither the surgeon input device nor any component of the central control system is in the service mode, since it is operating in the clinical mode. Thus, this time, the method moves to step 814/815 where the surgeon input device is enabled to control the surgical instrument/endoscope. There may be other conditions which must be fulfilled in order for the control system to approve the surgical instrument/endoscope being under the operative control of the surgeon input device. However, the control system will not prevent the control due to the service/clinical mode status of the surgeon input device controller or any component of the central control system.

The safety mechanisms described herein prevent the surgical robotic system from being used to perform surgery whilst any part of the surgical robotic system is in the service mode. Thus, there is no reliance on a service engineer remembering to power cycle the surgical robotic system in order to prevent the system from being used to perform surgery whilst in the service mode.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system configured to implement a safety mechanism of a surgical robot, the surgical robot operable in a clinical mode in which the surgical robot can perform surgery in response to inputs received at a surgeon input device of a remote surgeon console, the clinical mode comprising safety features, and the surgical robot operable in a service mode in which tests and diagnostics can be run on the surgical robot in response to inputs received from a service interface, the service mode mirroring the clinical mode except in that several of the safety features of the clinical mode are disabled, the control system configured to, in the safety mechanism:
receive a transition command to transition the surgical robot to a surgical clinical mode or a calibration clinical mode in which the surgical robot is calibrated for surgery;
in response to receiving the transition command, determine if the surgical robot is operating in the service mode; and
if the surgical robot is determined to be operating in the service mode, prevent the surgical robot from being transitioned to the surgical clinical mode or the calibration clinical mode.

2. The control system as claimed in claim 1, wherein the surgical robot comprises a surgical robot arm, the surgical robot arm comprising a series of joints by which its configuration can be altered, the series of joints extending from a base at a proximal end of the surgical robot arm to an attachment for a surgical instrument at a distal end of the surgical robot arm, the control system being configured to control the surgical robot arm in dependence on inputs received at the surgeon input device of the remote surgeon console, to alter the configuration of the surgical robot arm whilst maintaining an intersection between a surgical instrument attached to the surgical robot arm and a fulcrum, the control system configured to determine the position of the fulcrum in the calibration clinical mode.

3. The control system as claimed in claim 1, wherein the control system is configured to transition the surgical robot to the surgical clinical mode from the calibration clinical mode only upon successful calibration of the surgical robot arm in the calibration clinical mode.

4. The control system as claimed in claim 1, further configured to output a signal to the remote surgeon console for output by the remote surgeon console, the signal indicative that the surgical robot has been prevented from transitioning to the surgical clinical mode or the calibration clinical mode.

5. The control system as claimed in claim 1, further configured to output a signal to the surgical robot for output by the surgical robot, the signal indicative that the surgical robot has been prevented from transitioning to the surgical clinical mode or the calibration clinical mode.

6. The control system as claimed in claim 2, wherein the transition command is received via a user input on the surgical robot arm or via a user input on the remote surgeon console.

7. The control system as claimed in claim 1, further configured to, if the surgical robot is determined to not be operating in the service mode, enable the surgical robot to be transitioned to the surgical clinical mode or the calibration clinical mode.

8. The control system as claimed in claim 1, further configured to:
receive a power cycle command to power cycle the surgical robot;
in response to receiving the power cycle command, power cycling the surgical robot;
upon successfully power cycling the surgical robot, enabling the clinical mode; and
on subsequently receiving the transition command, enabling the surgical robot to be transitioned to the surgical clinical mode or the calibration clinical mode.

9. The control system as claimed in claim 8, wherein power cycling the surgical robot comprises performing power on self-test checks.

10. The control system as claimed in claim 8, wherein power cycling the surgical robot comprises loading the surgical robot in a predetermined configuration having predefined states.

11. The control system as claimed in claim 2, wherein the control system is distributed and comprises a surgical robot controller, a surgeon input device controller, and one or more additional system controllers, wherein the control system is configured to:
receive an engagement command to engage a surgical instrument;
in response to receiving the engagement command, determine if the surgeon input device controller is operating in the service mode; and
if the surgeon input device controller is determined to be operating in the service mode, prevent the surgeon input device controller from enabling the surgeon input device to control the surgical instrument.

12. The control system as claimed in claim 11, configured to:
in response to receiving the engagement command, determine if the one or more additional system controllers is operating in the service mode; and
if the one or more additional system controllers is determined to be operating in the service mode, preventing the one or more additional system controllers from enabling the surgeon input device to control the surgical instrument.

13. The control system as claimed in claim 11, further configured to output a signal to the remote surgeon console for output by the remote surgeon console, the signal indicative that the surgeon input device has been prevented from controlling the surgical instrument.

14. The control system as claimed in claim 11, further configured to output a signal to the surgical robot for output by the surgical robot, the signal indicative that the surgeon input device has been prevented from controlling the surgical instrument.

15. The control system as claimed in claim 11, further configured to, if the surgeon input device controller and/or the one or more additional system controllers is determined to not be operating in the service mode, enable the surgeon input device to control the surgical instrument.

16. The control system as claimed in claim 11, further configured to:
receive a power cycle command to power cycle the surgeon input device controller and/or the one or more additional system controllers;
in response to receiving the power cycle command, power cycling the surgeon input device controller and/or the one or more additional system controllers;
upon successfully power cycling the surgeon input device controller and/or the one or more additional system controllers, enabling the clinical mode; and
on subsequently receiving the engagement command, enabling the surgeon input device to control the surgical instrument.

17. The control system as claimed in claim 16, wherein power cycling the surgeon input device controller and/or the one or more additional system controllers comprises loading the surgeon input device controller and/or the one or more additional system controllers in a predetermined configuration having predefined states.

18. The control system as claimed in claim 11, wherein each controller of the distributed control system is operable in either the service mode or clinical mode independently of whether another controller of the distributed control system is in the service mode or clinical mode.

19. The control system as claimed in claim 1, wherein in the service mode, a safety feature of the clinical mode which is disabled is fault checking on communication links between a robot arm base controller located at the base of the robot arm and joint controllers located at the joints of the robot arm.

20. The control system as claimed in claim 1, wherein the surgical clinical mode and the calibration clinical mode are sub-modes of the clinical mode.
